(19) United States
(12) Patent Application Publication
(10) Pub. No.: US 2026/0185929 A1
(43) Pub. Date: Jul. 2, 2026

(54) Methods For Characterizing Fluids Flowing Through A Conduit With A System Including Optical Emitters And Detectors

(71) Applicant: Stryker Corporation, Portage, MI (US)

(72) Inventors: Stephen Faul, Cork (IE); Micheal Burke, Cork (IE); Mayank Kumar, San Jose, CA (US); Keng-Tsai Lin, San Jose, CA (US)

(73) Assignee: Stryker Corporation, Portage, MI (US)

(21) Appl. No.: 19/131,654

(22) PCT Filed: Nov. 21, 2023
(86) PCT No.: PCT/US2023/080666

§ 371 Date: May 21, 2025

Related U.S. Application Data
(60) Provisional application No. 63/426,909, filed on Nov. 21, 2022.

(51) Int. Cl. G01N 21/31; A61M 1/00; G01N 21/85; G01N 33/49

(52) U.S. Cl. CPC G01N 21/31; A61M 1/73; G01N 21/85; G01N 33/49; A61M 2205/3313

(57) ABSTRACT

Systems and methods for characterizing fluid through a conduit. The conduit may be noncircular, and a sensor module may include emitters and detectors spaced apart from one another by different distances such that light signals travel through the non-circular conduit in longer and shorter paths. The emitters may be alternatingly fired, or a second emitter may be fired if the light signal detected by a first detector is below a sensitivity threshold. More than two emitters and detectors may be provided for which a data rich matrix is provided based on absorbance and scatter values from each of the detectors in combination with each of the emitters. The travel paths and/or angles of the various combinations of emitters and detectors may be different. The data rich matrix may be provided to a machine-trained neural network to implement an algorithm to determine fluidic components within the fluid.

### CROSS-REFERENCE TO RELATED APPLICATIONS

The present application claims priority to and all the benefits of U.S. Provisional Patent Application No. 63/426,909, filed on Nov. 21, 2022, the entire contents of which are expressly incorporated herein by reference.

### BACKGROUND

Inaccurate characterization of fluids removed from a patient, such as during a surgical procedure, may put a patient's health at risk or unnecessarily consume medical resources. For example, where the fluid is blood, overestimation of blood loss results in the unnecessary consumption of transfusion-grade blood and may lead to unnecessary clinical risk to the patient. As another example, underestimation of blood loss may lead to delayed resuscitation and transfusion, increased risk of infections, tissue death, or even patient death, such as in the event of hemorrhage.

Certain existing fluid characterization systems utilize optical emitter-detector pairs being disposed opposite one another about a circular conduit. These systems are often deficient in instances where the fluid contains a high concentration of darker fluidic components that attenuate higher proportions of the light signals being transmitted through the fluid. An example of particular interest is high concentrations of blood within the fluid, wherein the light signals may be too attenuated upon reaching the optical detector to produce sufficiently accurate results for a sensitivity range of the optical detectors. One solution is to increase an intensity of the light from the light sources to ensure that sufficient light reaches the optical detector. However, increasing the intensity of the light may conversely be outside the upper sensitivity limit of the optical detectors in instances when the fluid contains little or no blood. Alternatively, adjusting the gain settings of the optical detectors is associated with similar shortcomings. In effect, the existing systems lack the detection range needed to characterize in real-time the fluids being quickly drawn through a conduit and containing high and low concentrations of blood in an unpredictable flow.

In detecting blood within the fluid, it is known to emit light at differing wavelengths, for example, infrared and visible green light. Yet another shortcoming of existing systems that utilize the emitter-detector pairs is limited data. In other words, the processor receives data from only a single detector per emitter-detector pair, from which only basic aspects of the fluid may be characterized. It would be desirable, however, for an improved fluid characterization system to generate more robust and rich data, from which more advanced processing techniques may be leveraged to perform a more detailed analysis of the fluids in real-time.

### SUMMARY

Among other aspects, the present disclosure provides high dynamic range (HDR) sensing to characterize fluids flowing within a conduit. The HDR sensing accommodates a wider range of concentrations of darker fluids (e.g., blood within) the conduit without requiring wider sensitivity ranges for optical detectors. It should be appreciated that the HDR sensing may be used in combination with other aspects of the present disclosure provided herein, for example, firing sequencing of optical emitters, scatter signal processing, multivariable analysis, and the like.

According to a first aspect, a method of characterizing fluids flowing through a non-circular conduit with a system including first and second optical emitters, first and second optical detectors, and a processor is provided. The method includes transmitting with the first optical emitter a first light signal through the non-circular conduit and the fluids along a first axis. Once the first light signal is sent through the conduit and the fluids, the first optical detector detects the first light signal that was at least partially absorbed by the fluids as the light signal traveled along the first axis through the fluids. Additionally, the method includes transmitting, with the second optical detector, a second light signal through the non-circular conduit along a second axis different than the first axis such that a relative path of one of the first and second light signals through the non-circular conduit is shorter than the other. As with the first light signal and the first optical detector, the second optical detector detects the second light signal that was at least partially absorbed by the fluids. Finally, the method includes determining a concentration of a fluidic component within the fluids based on the first and second light signals.

According to a second aspect, a method of characterizing fluids flowing through a conduit with a system including first and second optical emitters, first and second optical detectors, and a processor is provided. The method includes repeatedly transmitting, with the optical emitter, light signals through the conduit and the fluids. After the light signals have been transmitted, the light signals that were at least partially absorbed and scattered by the fluids are detected by each of the first and second optical detector. Further, the optical emitters and the first and second optical detectors are arranged in an array about the conduit such that distances between the optical emitter and each the first and second optical detectors are different. After the light signals pass through the fluid, the processor is used to determine an absorbance value and a scatter value from data from each of the first and second optical detectors for each of the light signals. Finally, a concentration of a fluidic component within the fluids based on the absorbance and scatter values.

According to a third aspect, a method of characterizing fluids flowing through a conduit with a system including first and second optical emitters, first and second optical detectors, and a processor is provided. The method begins with transmitting, with the first and second optical emitters, first and second light signal through the conduit and the fluids. After the light signals pass through the fluid and are at least partially absorbed and scattered by the fluids, the light signals are detected with each of the first and second optical detectors. In terms of arrangement, the first and second optical emitters and the first and second optical detectors are arranged in an array about the conduit such that distances between combinations of the first and second optical emitters and the first and second optical detectors are different. Further, the processor is used to determine an absorbance value and a scatter value from data from each of the first and second optical detectors for each of the first and second light signals to provide a data matrix and determine a concentration of a fluidic component within the fluids based on the data matrix.

According to a fourth aspect, a method of characterizing fluids flowing through a conduit with a system including optical emitters arranged in an array about the conduit, optical detectors arranged in the array about the conduit, and a processor is provided. The method includes transmitting, with the optical emitters, light signals through the conduit and the fluids. The optical emitters are sequentially activated, one at a time, in a positional order about the conduit. Afterwards, the light signals that were at least partially absorbed and scattered by the fluids are detected with the optical detectors. Finally, the processor determines an absorbance value and a scatter value from data from the optical detectors for each of the light signals and determines a concentration of a fluidic component within the fluids based on the absorbance values and scatter values.

According to a fifth aspect, a method of characterizing fluids flowing through a conduit with a system including optical emitters arranged in an array about the conduit, optical detectors arranged in the array about the conduit, and a processor is provided. The method includes transmitting, with a first of the optical emitters, light signals through the conduit and the fluids. Afterward, using one of the optical detectors adjacent or closest to the first optical emitter, the light signals that were at least partially absorbed and scattered by the fluids are detected. Finally, the processor determines an absorbance value and a scatter value from data from the optical detectors for each of the light signals and determines a concentration of a fluidic component within the fluids based on the absorbance values and scatter values.

According to a sixth aspect, a medical waste collection system is provided. The medical waste collection system includes a vacuum pump and a receptacle in fluid communication with the vacuum pump which is configured to collect fluids under influence of suction from the vacuum pump. The system also includes a sensor module which includes a housing, an emitter, and a detector. The emitter and the detector are positioned adjacent to an outer wall of, and external to, the receptacle, and the emitter is configured to emit light signals while the detector is configured to detect the light signals being absorbed and/or scattered by the fluid within the receptacle. Finally, the system includes a processor in electronic communication with the sensor module which is configured to receive sensor data from the detector and characterize a fluidic component of the fluid.

According to a seventh aspect, a sensor module for characterizing fluids from a patient is provided. The sensor module includes a housing which has a sidewall having longer sidelength and a shorter sidelength to collectively define a non-circular lumen. First and second light emitting diodes are coupled to the housing and are configured to output light signals of a first wavelength and a second wavelength, respectively. The second wavelength is lower than the first wavelength. Further, first and second detectors are coupled to the housing. The first detector is configured to detect the light signals of the first wavelength from the first light emitting diode and scattered light signals of the second wavelength from the second light emitting diode, while the second detector is configured to detect the light signals of the second wavelength from the second light emitting diode and scattered light signals of the first wavelength from the first light emitting diode. The second light emitting diode and the second detector are positioned on or adjacent to the sidewall on the shorter side length to be positioned across the major cross-sectional dimension.

According to an eighth aspect, an optical emitter for use with a sensor module for characterizing fluids from a patient is provided. The optical emitter includes an emitter configured to output light signals of multiple wavelengths. In order to output the light signals of multiple wavelengths, the sensor module includes a light guide coupling the optical emitter to each of a first and a second light emitting diode. The first light emitting diode is configured to output light signals of a first of the multiple wavelengths, while the second light emitting diode is configured to output light signals of a second of the multiple wavelengths. The lights signals travel from the light emitting diodes, along the light guide, and through the optical emitter.

According to a ninth aspect, a sensor module for characterizing fluids from a patient is provided. The sensor module includes a housing configured to be coupled to a non-circular conduit which has a first conduit seat arranged to be positioned adjacent to one side of the non-circular conduit when the housing is coupled to the non-circular conduit. The housing also includes a second conduit seat arranged to be positioned adjacent to an opposing side of the non-circular conduit when the housing is coupled to the non-circular conduit. More specifically, the first and second conduit seats define a lumen when the housing is coupled to the non-circular conduit. The lumen includes a minor cross-sectional dimension and a major cross-sectional dimension which is larger than the minor cross-sectional dimension. Aside from the housing, the sensor module further includes a first light emitting diode coupled to the housing which is configured to output light signals of a first wavelength, and a second light emitting diode coupled to the housing and configured to output light signals of a second wavelength that is different from the first wavelength. To detect light coming from the light emitting diodes, the sensor module also includes a first detector coupled to the housing opposite the first light emitting diode about the minor cross-sectional dimension and a second detector coupled to the housing opposite the second light emitting diode about the major cross-sectional dimension. The first detector being is configured to detect the light signals of the first wavelength from the first light emitting diode and scattered light signals of the second wavelength from the second light emitting diode, and the second detector is configured to detect the light signals of the second wavelength from the second light emitting diode and scattered light signals of the first wavelength from the first light emitting diode.

Any of the above aspects can be combined in part or in whole with any other aspect. Any of the above aspects, whether combined in part or in whole, can be further combined with any of the following implementations, in full or in part.

In order to provide distinct travel paths for the light signals from the light emitting diodes, the first and second axes along which the light signals are transmitted may be perpendicular to one another. The first axis and the second axis may be transverse to a longitudinal axis of the non-circular conduit. The first and second axes may correspond to a respective one of a major cross-sectional dimension and a minor cross-sectional dimension of the non-circular conduit. The non-circular conduit may be elliptical, oval, hexagonal, octagonal, or rectangular.

To avoid mixing signals, the step of transmitting the second light signal may be performed after the step of transmitting the first light signal such that only a singular one of the first optical emitter and the second optical emitter is operable at a time. To that end, the method may include alternating the steps of transmitting the first light signal and the second light signal. The step of alternating the steps may be performed continuously and repeatedly during operation of the system.

The method may include transmitting the first light signal at a first wavelength; and transmitting the second light signal at a second wavelength different than the first wavelength. The method may also include comparing with the processor the first light signal against a sensor sensitivity threshold and performing the step of transmitting the second light signal in response to the first light signal being below the sensor sensitivity threshold.

The step of determining the concentration of the fluidic component may further include analyzing the first and second light signals with a parametric model generated by a machine-trained neural network.

The method may include generating with the second optical detector a first scattered light value indicative of the first light signal being at least partially scattered by the fluids, generating with the first optical detector a second scattered light value indicative of the second light signal being at least partially scattered by the fluids, and determining with the processor the concentration of the fluidic component further based on the first and second scattered light values.

The optical emitters and the optical detectors may be arranged in an array about the conduit such that distances between combinations of the first and second optical emitters and the first and second optical detectors are different. The optical emitters and the optical detectors may be coupled to an outer diameter of the conduit to form a ring. The optical detectors may be arranged at different angles relative to each of the optical emitters.

The sensor module may further include a processor in communication with at least one of the first light emitting diode, the second light emitting diode, the first detector, and the second detector. The processor may be configured to determine a concentration of a fluidic component of fluid flowing through the non-circular conduit based on the light signals as detected by the first and second detectors and the scattered light signals as detected by the first and second detectors. To that end, the processor may be configured to determine the concentration of the fluidic component of the fluid flowing through the non-circular conduit by analyzing the light signals and the scattered light signals with a parametric model generated by a machine-trained neural network. The processor may also be configured to determine an absorbance value and a scatter value from data from each of the first and second optical detectors for each of the first light signal, the second light signal, and the scattered light signals to provide a data matrix, and determine a concentration of a fluidic component within fluid flowing through the non-circular conduit based on the data matrix of the absorbance values and scatter values.

Finally, the method may include displaying the concentration of a fluidic component on a display.

### BRIEF DESCRIPTION OF THE DRAWINGS

Advantages of the present invention will be readily appreciated as the same becomes better understood by reference to the following detailed description when considered in connection with the accompanying drawings.

FIG. 1A is a representation of a fluid characterization system in which a sensor module is coupled to a conduit. The conduit may be coupled to a manifold configured to be received within a medical waste collection system. Additionally or alternatively, the suction tube may be coupled to a cartridge configured to be received within a console.

FIG. 1B is a schematic representation of the fluid characterization system of FIG. 1A including a suction device, the conduit, the sensor module, a receptacle, and a vacuum source.

FIG. 2A is a perspective view of the sensor module of FIGS. 1A and 1B.

FIG. 2B is a sectional view of the sensor module depicted in FIG. 2A.

FIG. 2C is a sectional view of another implementation of the sensor module depicted in FIG. 2A.

FIG. 3A is a sectional view of the sensor module depicted in FIG. 2B schematically representing positions of optical emitters and optical detectors disposed about a non-circular conduit.

FIG. 3B is a sectional view of the sensor module depicted in FIG. 2C schematically representing positions of the optical emitters and the optical detectors disposed about another non-circular conduit.

FIGS. 4A and 4B are schematic views in which light signals are emitted from optical emitters, through non-circular conduits, and received by optical detectors.

FIGS. 5A and 5B are schematic views in which light signals passing through non-circular conduits may be detected to determine a fill level of the fluid within the conduit.

FIG. 6 is a schematic view illustrating light signals as well as scatter signals resulting from the light signals passing through a non-circular conduit.

FIG. 7A is a sectional view of the sensor module in which a “one to many” arrangement is schematically represented.

FIG. 7B is a sectional view of the sensor module in which a “many to one” arrangement is schematically represented.

FIGS. 7C and 7D are sectional views of the sensor module in which a “many to many” arrangement is schematically represented. FIG. 7C shows the conduit as non-circular, and FIG. 7D shows the conduit as circular.

FIG. 8 is a representation of a receptacle of the medical waste collection system of FIGS. 1A and 1B with the sensor module coupled thereto such that the emitter and the detector are positioned adjacent an outer wall of the receptacle and external to the receptacle.

FIG. 9 is a representation of an emitter including light emitting diodes of differing wavelengths, and a light guide.

### DETAILED DESCRIPTION

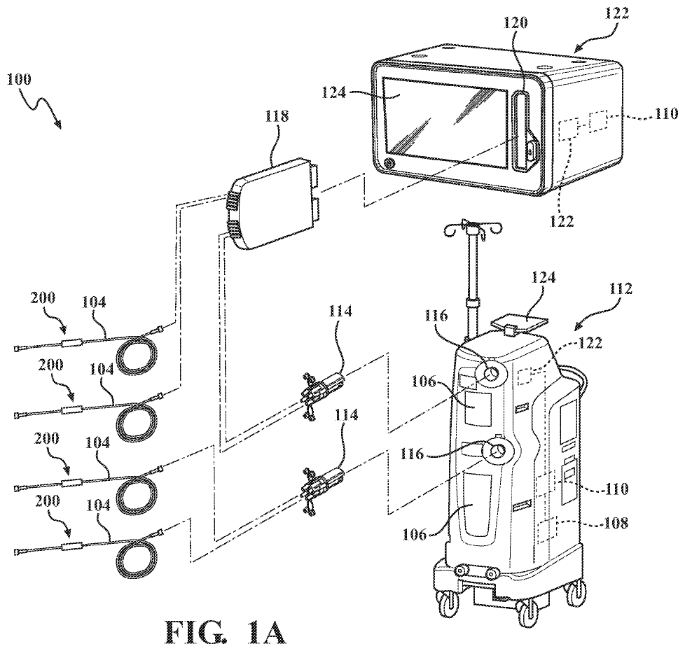

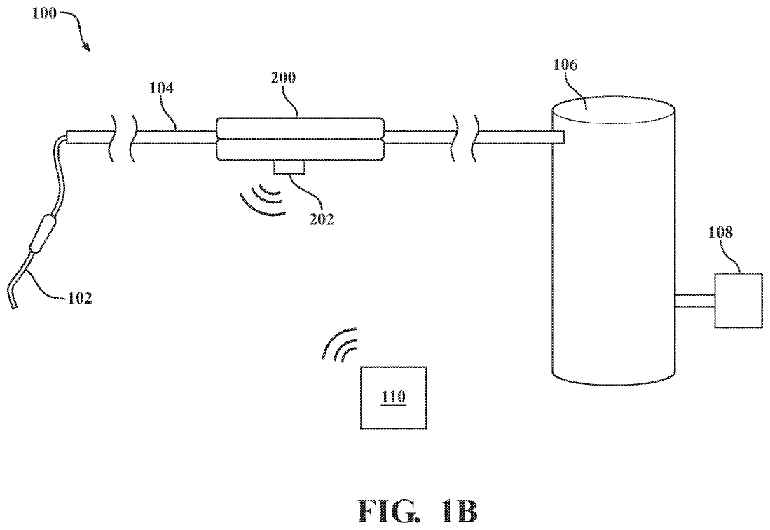

FIGS. 1A and 1B show a fluid characterization system 100 for characterizing fluids removed and collected from a patient. The system 100 includes a conduit 104, a receptacle 106, a vacuum source 108, and a sensor module 200. The removed patient fluids may be drawn through the conduit 104 to be collected in the receptacle 106 under influence of suction from the vacuum source 108. As shown, the receptacle 106 and the vacuum source 108 may be integrated on a mobile rover of a medical waste collection system 112, such as those sold under the tradename Neptune by Stryker Corporation (Kalamazoo, Mich.) and disclosed in commonly-owned U.S. Pat. No. 7,621,898, issued Nov. 24, 2009, the entire contents of which are hereby incorporated by reference. By being coupled to the conduit 104, the sensor module 200 can be integrated onto legacy waste management systems without extensive equipment additions or modifications. For example, the conduit 104 may be coupled upstream a manifold 114 configured to be removably received in a receiver 116 of the medical waste collection system 112. Additionally or alternatively, the conduit 104 may be coupled to a cartridge 118 configured to be removably received in a receiver 120 of a console 122. The implementation shown in FIG. 1A is exemplary, and FIG. 1B shows a schematic representation of another system 100 with like numerals indicating like components. The receptacle 106 and the vacuum source 108 of FIG. 1B may be, for example, integrated within a medical facility, and a fluid retrieval device 102 (e.g., a suction wand) may be provided.

The system 100 includes the sensor module 200 configured to be coupled to the conduit 104. The sensor module 200 includes one or more sensors 230 and/or other measurement devices for detecting one or more optical properties of the fluid passing through the conduit 104. The system 100 may further include at least one processor 110 in electronic communication with and receiving sensor data from the one or more sensors 230. The processor 110 may be integrated with the sensor module 200 (e.g., within a sensor housing 220), and/or the sensor data may be communicated by a wireless transceiver 202 of the sensor module 200 to a complementary transceiver on the medical waste collection system 112 and/or the console 122 including the processor 110 or another processor. The processor 110 is configured to execute computer-implemented instructions stored on non-transitory memory (not shown) to analyze or characterize the fluids. Of interest is determining fluidic components of the fluids, in particular blood concentration. From the determined blood concentration, blood loss may be estimated or quantified based on a determined collected volume of the fluids as measured by a fluid measuring subsystem within the receptacle 106 of the medical waste collection system 112. Additionally or alternatively, the blood loss may be estimated or quantified based on a determined volumetric flow rate per unit time, such as measured by ultrasonic sensors (not shown), a camera, mass flow sensors, or other electronics of the sensor module 200. The medical waste collection system 112 and the console 122 may include a display 124 for displaying results of the analysis of the fluids in real-time, for example, the average or cumulative blood loss from the patient.

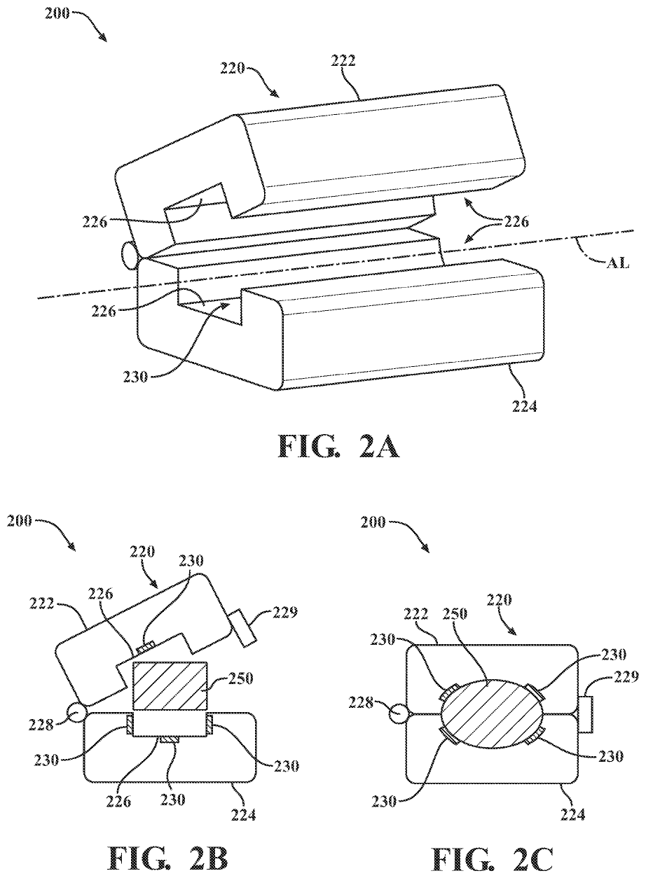

Referring to FIGS. 2A-2C, exemplary forms of the sensor module 200 are depicted. The sensor module 200 may be coupled to or at least partially disposed in a housing 220 configured to couple the sensor module 200 to the conduit 104. Alternatively, at least some of the sensors 230 and other components of the sensor module 200 may be separately coupled to the conduit 104 (e.g., outside of a single common housing). The sensor module 200 may be adjustable or universal such that the housing 220 may be coupled to a wide range of conduit types (e.g., without reliance on being coupled to any specific type or brand of conduit).

The housing 220 may be configured to clamp onto the conduit 104. For example, as shown in FIG. 2A, a housing 220 for the sensor module 200 may include at least a first jaw 222 and a second jaw 224, which together are configured to clamp onto the conduit 104. Each jaw 222, 224 may include at least one conduit seat 226 shaped and sized to receive the conduit 104. For example, for the conduit 250 being a circular in cross-section, each jaw 222 or 224 may include the conduit seats 226 that are semicircular. For implementations in which the conduit 250 is non-circular, the conduit seats 226 may collectively form a rectangle, an oval, a hexagon, an octagon, or other suitable geometry to accommodate the conduit 250. Furthermore, the housing 220 may include three, four, or other suitable numbers of jaws sized and shape to accommodate the non-circular conduit. In an alternative implementation, the housing 220 may be C-shaped and configured to clip onto the non-circular conduit 250. One or more of the conduit seats 226 may include a deformable surface configured to compress such that the housing 220 can receive and conform to non-circular conduits. In still other variations, the sensor module 200 may be packaged with a plurality of conduit seats 226, each of different size and shape to accommodate non-circular conduits of varying geometries.

The conduit seat 226 engages the conduit 250 to maintain a relative position of the one or more sensors 230 about the conduit 250. When the housing 220 is clamped onto the conduit 250, the conduit seats 226 collectively form a lumen defining a longitudinal axis AL as shown in FIG. 2A. Therefore, the longitudinal axis AL is substantially parallel to the conduit 250, and the sensors 230 may be disposed radially about the longitudinal axis AL and the conduit 250.

The sensors 230, generically shown in FIGS. 2B and 2C, may be further defined as optical emitters 232 and optical detectors 234 as used hereinafter. As implied by their names, the optical emitters 232 are configured to emit light signals, and the optical detectors 234 are configured to detect the light signals emitted from optical emitters 232. The detectors 234 generate and transmit data to the processor 110 for characterizing a fluid flow (e.g., estimate a velocity of the flow, a mass flow rate of the flow, a volume flow rate of the flow, or any suitable combination thereof), for estimating concentration(s) of one or more fluidic components, or both.

Existing systems may include emitter-detector pairs, the emitter of each of the emitter-detector pairs being arranged diametrically opposite a circular conduit from the corresponding detector. As such, a travel path from the emitter, through the conduit, and to the corresponding detector is the same for all of the emitter-detector pairs. As mentioned, the intensity of the light signals may be attenuated by the fluid flowing through the conduit, such as blood in the fluid absorbing and/or scattering the light signals. For the sensitivity range for most detectors, however, the existing systems are unable to characterize the fluids accurately in procedures in which there may be high and low concentrations of blood in a rapidly changing manner as the fluid is drawn through the conduit 250. For example, high concentrations of blood may result in the light signals being too attenuated to be detected by the detector.

The system 100 of the present disclosure overcomes such shortcomings by providing high dynamic range (HDR) sensing, and further providing more robust data to the processor 110. To that end, the sensor module 200 may be coupled to a non-circular conduit, and the emitters and detectors 232, 234 are positioned about the non-circular conduit in manners to be described.

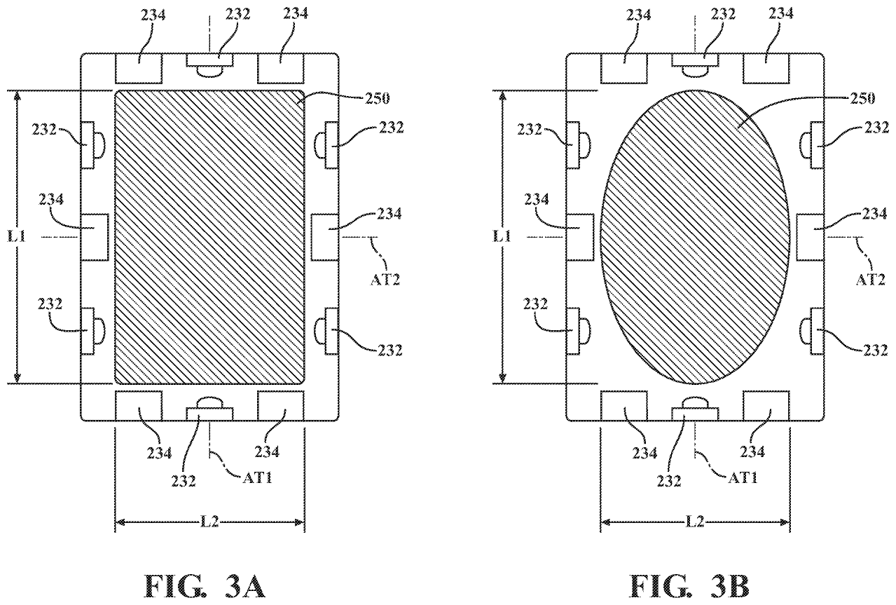

Referring now to FIGS. 3A and 3B, exemplary non-circular cross-sections of the conduit 250 are depicted. The conduit 250 includes at least two distinct sidelengths—a first sidelength L1 and a second sidelength L2. The longer sidelength may be referred to as a major cross-sectional dimension, while the shorter sidelength may be referred to as a minor cross-sectional dimension. In the illustrated embodiment, the first sidelength L1 is the major cross-sectional dimension and the second sidelength L2 is the minor cross-sectional dimension. The conduit 250 may be substantially rectangular, elliptical, or oval, or another suitable geometry. Alternatively, the conduit 250 may be initially formed with a circular cross section and deformed or compressed by the conduit seats 226 to become non-circular. FIGS. 3A and 3B schematically represent exemplary positioning of the emitters 232 and the detectors 234 about the conduit 250 with a remainder of the sensor module 200 omitted for clarity of depiction.

The sensor module 200 generally defines two axes, such a first transverse axis AT1 and a second transverse axis AT2. The transverse axes AT1, AT2 may be perpendicular to one another and perpendicular to the longitudinal axis AL, or any two axes nonparallel to one another and nonparallel to the longitudinal axis AL. The emitters 232 and the detectors 234 of FIGS. 3A and 3B may be arranged at the same axial position along the conduit 250. Alternatively, the emitters 232 may be axially spaced along the conduit 250 relative to each other and/or to the detectors 234, in which case the transverse axes AT1, AT2 may not be perpendicular to the longitudinal axis AL. There may be additional sets of the emitters 232 and the detectors 234 axially spaced along the conduit 250, in addition to the radial arrangements disclosed herein.

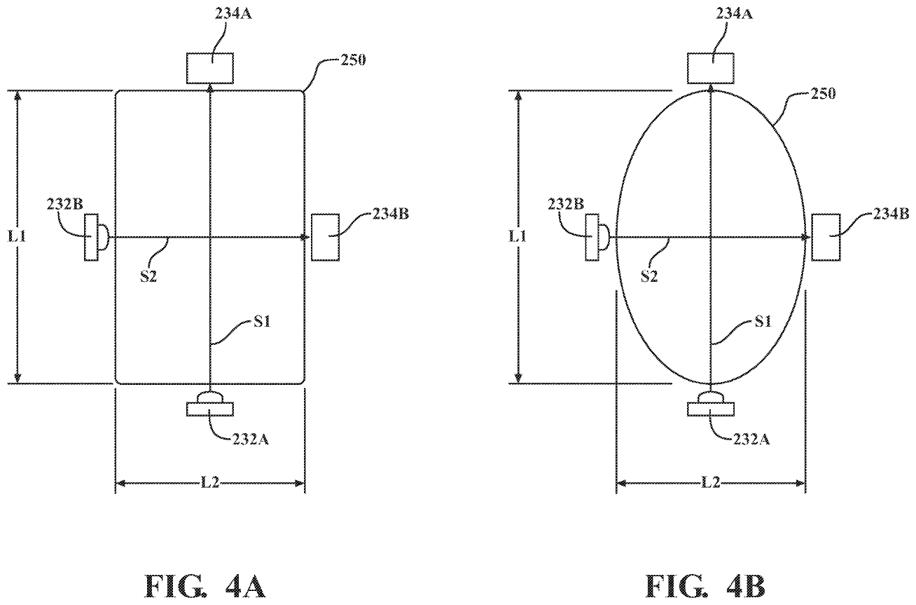

Referring to FIGS. 4A and 4B, the cross-sections of the conduit 250 are depicted along with representations of the light signals being transmitted from the emitters 232 to the detectors 234. The light signals are illustrated as a first light signal S1 and a second light signal S2. For illustrative purposes, the light signals S1, S2 are represented as transmitted substantially along the first and second transverse axes AT1, AT2, respectively, which are substantially parallel with the sidelengths L1, L2 of the conduit 250. Thus, the travel paths of each of the light signals S1, S2 are of different lengths. The travel path of the first light signal L1 is greater or longer than the travel path of the second light signal L2. For example, the travel path of the first light signal S1 may be at least 1.25, 1.5, 2 or more times longer than as the travel path of the second light signal S2. Other magnitudes are contemplated based on an aspect ratio of the non-circular conduit 250.

Since the travel paths of the light signals S1, S2 are of different lengths, multiple benefits are realized. First, the sensor module 200 provides for the HDR sensing to accommodate high and low concentrations of blood within the sensitivity ranges for the detectors 234. As mentioned, the fluidic content may vary between a high blood concentration and a low blood concentration, resulting in attenuation of the light signals from the absorption and/or scattering by the fluid. The operating parameters of the emitters 232 and the detectors 234 may specifically tuned in view of the relative magnitude between the first and second travel paths S1, S2 so that, regardless of the blood concentration within the fluids, a sufficient intensity of light is detectable by at least one of the detectors 234. More particularly, the intensity of the light emitted by the emitters 232 (or gain of the detectors 243) may be tuned or selected in view of the differing sidelengths L1, L2 such that (i) with high blood concentration (e.g., greater than 95%), the second light signal S2 travelling the shorter travel path is above the lower sensitivity limit of the detectors 234 even after it has been attenuated by the fluid, and (ii) with low blood concentration (e.g., 0%), the first light signal S1 is lower than the upper sensitivity limit of the detectors 234 even after it has been (or failed to be) attenuated by the fluid. While an inner diameter of a circular conduit could conceivably be narrowed to remain sensitive to high blood concentrations, the resulting cross-sectional area of the circular conduit would be too small to also be sensitive to low blood concentrations. The same is true for a wider circular conduit, as the wider circular conduit would remain sensitive to low blood concentrations but loss sensitivity to high blood concentrations. Using the non-circular conduit 250 overcomes these challenges.

It is contemplated the emitters 232 may emit the light signals and different intensities, and/or the detectors 234 may have different sensitivity ranges. For one example, the first emitter 232A configured to emit the first light signal S1 along the longer travel path may be brighter than the second light signal S2 emitted by the second emitter 232B. For another example, the first detector 234A configured to detect the first light signal S1 along the longer travel path may be more sensitive than the second detector 234B.

The detectors 234 detect the light signals, and generate data or signal values. The signal values are transmitted to the processor 110, from which the processor 110 may determine a concentration of a fluidic component, for example a hemoglobin concentration. Therefore, exemplary methods may include transmitting with the first emitter 232A a first light signal S1 through the non-circular conduit 250 and the fluids along the first axis AT1, and detecting the first light signal S1 with the first detector 234A. The first light signal S1 may have been at least partially absorbed and/or scattered by the fluids. Likewise, the second light signal S2 is transmitted through the non-circular conduit 250 with the second detector 232B along the second axis AT2, and detected by the second detector 234B. The second light signal S2 may have been at least partially absorbed and/or scattered by the fluids.

In certain implementations, the second light signal S1 is transmitted after the first light signal S1 is transmitted such that only a singular one of the first emitter 232A and the second emitter 232B is operated, activated, or “fired” at a time. In other words, the first and second light signals may be fired in an alternating manner. The alternate firing may be performed continuously and repeatedly during operation of the system, or in response to determined criteria, or a combination thereof. In one example, the alternate firing may be performed at fixed or varied time intervals. For another example, one of the first and second emitters 232A, 232B may be fired at a first fixed time interval, and the other fired at a second time interval different than the first fixed time interval.

In certain implementations, only one of the first and second emitters 232A, 232B is fired repeatedly with the other emitter being “idle.” The other emitter is fired only in response to the processor 110 determining a lack of light signals or other detectable characteristic being sensed by the detector(s) 234. For example, the first emitter 232A may be fired at a fixed or varied time interval, and the first detector 234A detects the first light signals S1. The processor 110 may compare the first light signals S1 against a predetermined sensor sensitivity threshold. If the processor 110 determines that the first light signals S1 have decreased below the sensor sensitivity threshold (e.g. if the first detector 234A does not detect the first light signals S1), the processor 110 may operate the second emitter 232B to begin firing at fixed or varied time intervals to compensate for the same. The sensor sensitivity threshold may be an instantaneous threshold, or an average threshold over a predetermined period of time (e.g., three seconds). In this respect, the system 100 may compensate in real-time for markedly fluctuating blood concentrations within the fluids.

In certain implementations, the first emitter 232A may emit the first light signal S1 of a first wavelength, and the second emitter 232B may emit the second light signal S2 of a second wavelength. The second wavelength is different from the first wavelength. For example, a first emitter may be an infrared light emitting diode (LED), and the second emitter may be a visible-light LED, such as a green LED. The infrared LED may be configured to emit light having a wavelength approximately in the range of 700 nanometers (nm) to 1000 nm, and more particularly within the range of 750 nm to 850 nm, and even more particularly within the range of 770 nm to 810 nm. The visible-light LED may be configured to emit light having a wavelength approximately in the range of 400 nm to 600 nm, and more particularly within the range of 550 nm to 600 nm, and even more particularly within the range of 570 nm to 580 nm.

It is understood that lower wavelength light scatters more readily than higher wavelength light, and therefore detecting the higher wavelengths at greater distances may be challenging. The sensor module 200 addresses such concerns by relating the wavelengths of the emitters 232 to the varying sidelengths L1, L2 of the conduit 250 (via the sensor housing 220). More particularly, for example, lower wavelength LEDs may be placed across the narrower sidelength L2 of the conduit 250 and higher wavelength LEDs may be placed across the wider sidelength L1 of the conduit 250. As such, the light signals S1, S2 may include different frequencies of light and the processor 110 may characterize the fluidic content based on the effect of the fluidic content on the different frequencies of light (e.g., via the Beer-Lambert Law). In other words, the processor 110 may be configured to correlate the signal values with the wavelengths of the light signals S1, S2, which provides more robust data from which the algorithms being implemented by the processor 110 may be used to characterize the fluidic content.

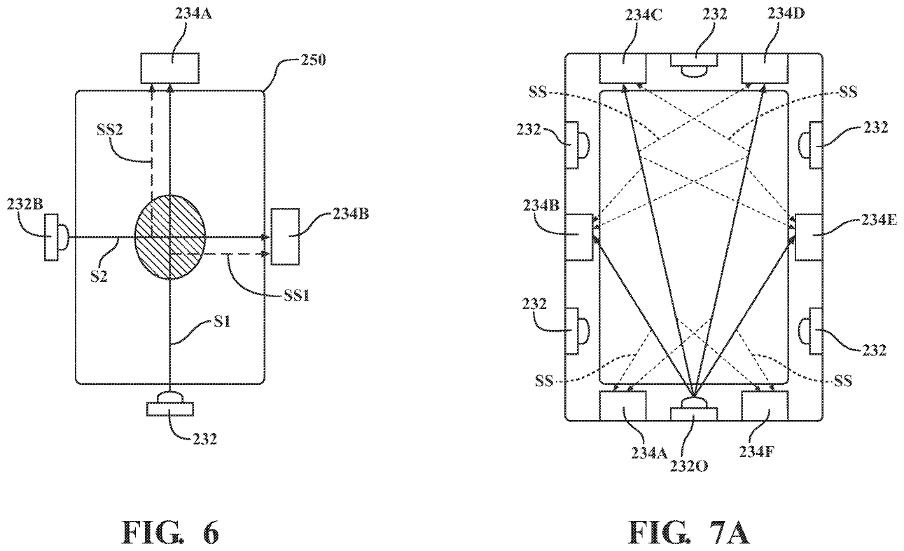

In addition to absorbance of the light signals, the light signals may also be scattered by particles in the fluid. For example and with reference FIG. 6, the light signals S1, S2 are shown passing through an illustrative particle of the fluid flowing through the conduit 250. First and second scatter signals SS1, SS2 are shown corresponding to each light signal S1, S2. The scatter signals SS1, SS2 result from a portion of the respective signal S1, S2 being scattered by the particle. The detectors 234 may be configured to receive one of the light signals S1, S2 as well as one of the scatter signals SS1, SS2. More particularly, the second detector 234B may receive the first scatter signal SS1 (in addition to the second light signal S2), and the first detector 234A may receive the second scatter signal SS2 (in addition to the first light signal S2). These light signals S1, S2 and scatter signals SS1, SS2 may be correlated to the differing wavelengths (and/or firing timing of the emitters 232) such that the processor 110 is configured to detect whether the signals being received is a light signal or a scatter signal. The signal values of the signals S1, S2, SS1, SS2 as received by the corresponding detectors 234 may be transmitted to the processor 110 to provide an even richer data matrix. The processor 110 may be configured to determine the fluid characteristics of the fluidic content based on the light signals S1, S2 received by the respective first and second detectors 234A, 234B, as well as the scatter signals SS1, SS2 received by the respective second and first detectors 234B, 234A. Therefore, exemplary methods may include generating with the second detector 232B the first scattered light value SS1 indicative of the first light signal S1 being at least partially scattered by the fluids. The second scattered light value SS2 indicative of the second light signal S2 being at least partially scattered by the fluids is generated with the first detector 234A. The fluidic component is determined with the processor 110 further based on the first and second scattered light values SS1, SS2.

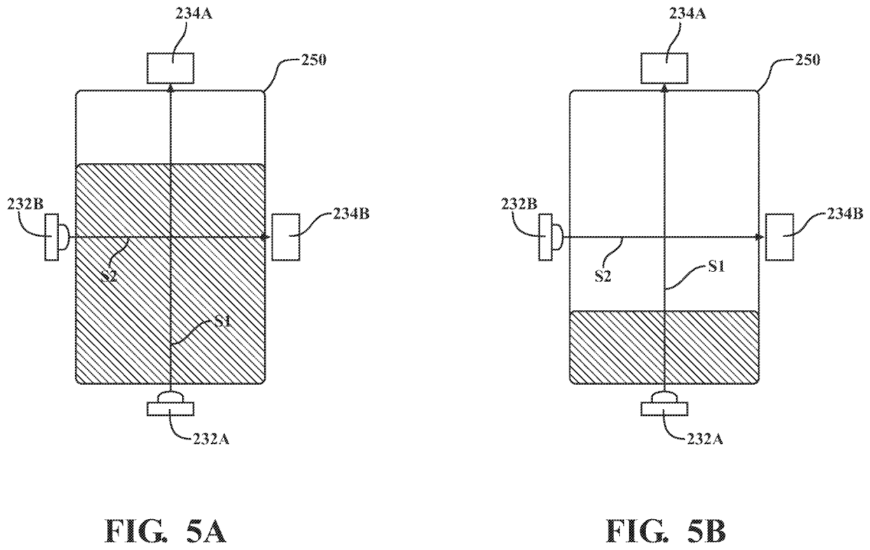

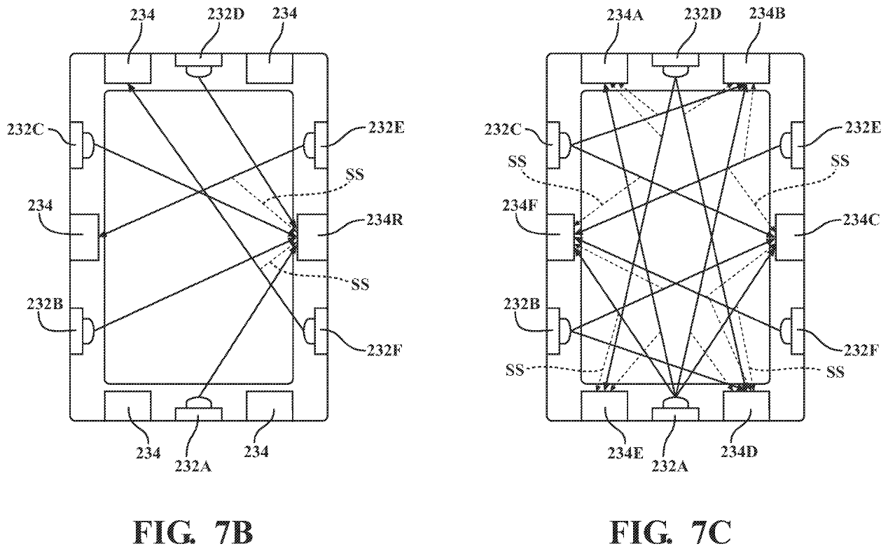

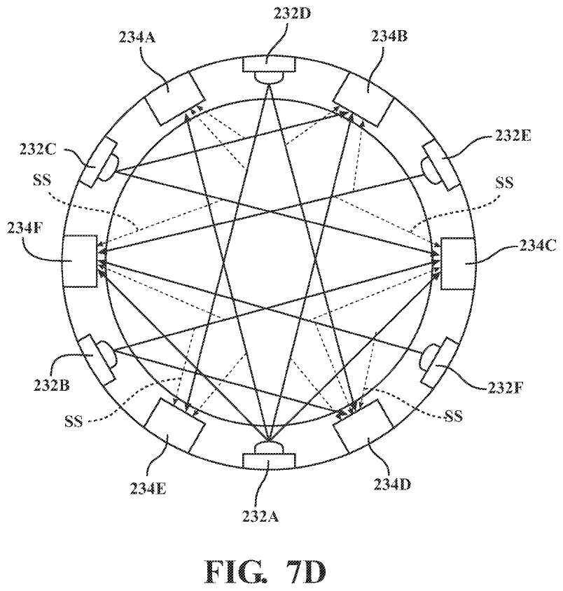

Additionally or alternatively, the light signals S1, S2 may be used to determine a fill level within the conduit 250. For illustrative purposes, the first transverse axis AT1 of FIGS. 3A and 3B may be oriented perpendicular to gravity while the second transverse axis AT2 may be oriented parallel to gravity. With further reference to FIGS. 5A and 5B, the conduit 250 is shown partially filled with different levels of fluid according to the shaded portions of the conduit 250. It is understood that, at higher levels of suction from the vacuum source 108, the fluid may not practically be “settled” within a lower portion of the conduit 250. Yet the light signals S1, S2 may still be attenuated and scattered even within highly irregular flow paths through the conduit 250 such that the principles herein remain applicable. Starting with FIG. 5A, since the fill level is above the travel path of the second light signal S2, the second light signal S2 may attenuated accordingly. The processor 110 may be configured to correlate the respective data from the first and second detectors 234A, 234B to estimate the fill level. On the other hand, FIG. 5B shows a lower fill level in which little to no attention of the second light signal S2 will occur (and the first light signal S1 will be attenuated less relative to FIG. 5A). Again, the fluid characterization algorithm executed by the processor 110 may be configured to correlate the respective data from the first and second detectors 234A, 234B to estimate the fill level. In implementations with additional emitters 232 and detectors 234 disposed about the conduit 250 (see FIGS. 7A-7D), additional data (e.g., attenuation and scatter data) from combinations of each of the detectors 234 and each of the emitters 232 may provide the data rich matrix for the processor 110 to determine a proportion of the conduit 250 that is filled with fluid.

The previous implementations described herein have included two emitter-detector pairs (i.e., 232A-234A and 232B-234B). In exemplary implementations, the sensor module 200 may include more than two emitters 232, and more than two detectors 234. The sensor module 200 may include three, four, six (see FIGS. 3A, 3B, and 7A-7D), ten or more of each of the emitters 232 and detectors 234. It is further contemplated that the sensor module 200 may include more emitters 232 than detectors 234, or more detectors 234 than emitters 232. FIGS. 7A-7D show exemplary arrangements of the emitters 232 and the detectors 234 with representations of various light signals and scatter signals being transmitted through the conduit and the fluid (removed for clarity). Not all of the light signals and scatter signals are illustrated for clarity.

Referring to FIG. 7A, a “one-to-many” arrangement is depicted in which an originating emitter 2320 emits the light signals configured to be received by more than one of detectors 234. The originating emitter 2320 may be configured to emit light signals each directed to a specific detector 234. For example, the originating emitter 2320 may be configured to emit light signals at a plurality of wavelengths of light, and each of the detectors 234 may be configured to receive one of the plurality of wavelengths of light. Alternatively, the originating emitter 2320 may be configured to emit the light signal which is configured to be received by all of the detectors 234 in a near-simultaneous manner.

It is appreciated from FIG. 7A that the travel paths between the emitters 232 and most or all of the detectors 234 are different. Moreover, the detectors 234 are located at different angles relative to the emitters 232. For instance, the originating emitter 2320 emits the light signals received by the various detectors 234B, 234C, 234D, 234B. The two detectors 234C, 234D disposed opposite the originating emitter 2320 effectively measure absorbance of the light signals through the entirety of the fluid. By contrast, the two detectors 234B, 234E measure absorbance of the light signals through a shorter travel path. Further, the scatter signals SS are also detected by the detectors 234. In this case, an array of sensor data, D, collected at regular intervals (in the region of hundreds of Hertz to capture changes in the fluid in flow), may be represented by:

$$D = \begin{matrix}S 1 \\ S 2 \\ S 3 \\ ⋮ \\ Sy\end{matrix}$$

where Si is the signal from the emitter 232 as received by the detectors 234, and y is the number of detectors 234. Alternatively, the above concept maybe applied to an implementation with a single emitter 232 and multiple detectors 234.

Therefore, certain exemplary methods may therefore include repeatedly transmitting, with the emitter 232, light signals through the conduit 250 and the fluids. The light signals that were at least partially absorbed and scattered by the fluids are detected with each of the first and second detectors 234. The emitters 232 and the first and second detectors 234 are arranged in an array about the conduit 250 such that distances between the emitter and each the first and second detectors are different. The processor determines an absorbance value and a scatter value from data from each of the first and second detectors for each of the first and second light signals. The processor 110 determines a concentration of a fluidic component within the fluids based on the absorbance values and scatter values.

Referring to FIG. 7B, a “many-to-one arrangement” is depicted with a plurality of emitters 232 each emit a light signal to be received by the same receiving detector 234R. In the many-to-one arrangement, the receiving detector 234R may be configured to receive a plurality of wavelengths of light, and the emitters 232 may be configured to each emit the light signal corresponding to a specific wavelength. Alternatively, each of the emitters 232 may be configured to transmit a light signal at a different time. The processor 110 is configured to correlate the signal value from the receiving detector 234R at a specific time to a singular one of emitters 232.

Referring to FIGS. 7C and 7D, a “many-to-many” arrangement is depicted with a plurality of emitters 232A-232F each emit multiple light signals received by each of the plurality of detectors 234A-234F. The light signals may be differentiated based on wavelength, timing, or any other signal characteristic. Thus this combination of light sources and sensors provides an exemplary rich data array of absorbance and scatter information from many wavelengths of light. Stated differently, the arrangement may include all combinations of emitter-detector pairs providing a signal value for the light signals, and all combinations of the emitter-detector pairs providing another signal value for the scatter signals. For example, each emitter 232 could be illuminated in turn, which each detector 234 measuring absorbance and scatter from that emitter 232. For instance, where Si corresponds to the i^{th} signal as received by the it detector 234 and Li corresponds to the i^{th} originating emitter 232, the pattern starts with L1 illuminated and measurements being recorded from S1, S2 and so on up to S5. L1 is then deactivated and L2 is illuminated and measurements are once again recorded from all sensors. This sequence continues until each light source has been illuminated and sensor data recorded. Once all light sources have been illuminated, the sequence starts again with the first light source. This sequence repeats at regular intervals in the region of hundreds of Hertz in order to provide timely updates of fluid changes in flow. Each iteration of this sequence produces a matrix of data D′ where:

$${D}^{′} = [ \begin{matrix}L 1 S 1 & L 2 S 1 & ⋯ & LxS 1 \\ L 1 S 2 & L 2 S 2 & ⋱ & LxS 2 \\ ⋮ & ⋱ & ⋱ & ⋮ \\ L 1 Sy & L 2 Sy & ⋯ & LxSy\end{matrix} ]$$

where Li is respective emitter (e.g., 232A-232F), Si is detector (e.g., 234A-234F), x is the number of emitters and y is the number of detectors 234. The matrix of data may contain columns corresponding to the detectors 234 and rows corresponding to the emitters 232. For example, the first column of data may include signal strengths associated with a first emitter 232 as received by each of the detectors 234. Further, the first row of data may include signal strengths of signals from each emitter 232 as received by one of the detectors 234.

The rich data matrix is provided to the processor 110 with which to characterize the fluidic content flowing through the conduit 250. In other words, the processor 110 may perform mathematical operations on the rich data matrix to characterize the fluidic content. A model, such as a neural network, Gaussian regression model, or other machine learning model, trained with representative test data, can use this matrix of information to determine the characteristics (e.g. blood concentration) of the fluid in the tube at each time interval. The processor 110 may determine concentration from the absorbance through the Beer-Lambert Law, for which there is a linear relationship between the absorbance of a solution and its concentration. It is understood that the light signals may have additional, distinct signal characteristics. The signal characteristics may be provided additional data to the processor 110 for characterizing the fluidic content. Furthermore, it should be appreciated that the implementations of multivariable analysis described with reference to FIGS. 7A-7D may be used with a conduit of a noncircular cross section (FIGS. 7A-7C) or a conduit of a circular cross section (FIG. 7D).

Certain exemplary methods may therefore include transmitting, with the first emitter, a first light signal through the conduit and the fluids; transmitting, with the second emitter, a second light signal through the conduit and the fluids; detecting, with each of the first and second detectors, the first and second light signals that were at least partially absorbed and scattered by the fluids, wherein the first and second emitters and the first and second detectors are arranged in an array about the conduit such that distances between combinations of the first and second emitters and the first and second detectors are different; determining with the processor an absorbance value and a scatter value from data from each of the first and second detectors for each of the first and second light signals to provide a data matrix; and determining with the processor a concentration of a fluidic component within the fluids based on the data matrix of the absorbance values and scatter values.

Even more information could be added to this matrix by illuminating the emitters 232 in differing sequences, patterns, groupings, or the like. In one example, the emitters 232 are sequentially activated, one at a time, in a positional order about the conduit (e.g., clockwise or counterclockwise). In another example, a first subset of emitters 232 are activated in combination, followed by a second subset of the emitters 232. The emitter 232 may also be illuminated at a specific frequency—the frequency may apply to any pattern of illumination. For example, the emitters 232 may be illuminated at regular intervals, such as in the region of hundreds of Hertz. Each iteration of illumination may provide the processor 110 with the rich data matrix with which to characterize the fluidic content. The timing of the light signals may also be controlled by the processor 110 based on data received from the detector(s) 234. For example, the processor 110 may cause the first light signal S1 to be emitted at a first time and cause the second light signal S2 to be emitted at a second time only if the first light signal S1 is not received by the detector 234 such that adequate information is provided to the processor 110 to characterize the fluidic content. More specifically, the processor 110 may determine the signal strength of the first light signal S1 as received by one of the detectors 234 and compare the signal strength against the sensor sensitivity threshold, the sensor sensitivity threshold corresponding to the lowest signal strength needed to characterize the fluidic content. If the first light signal S1 is not received with a signal strength above the sensor sensitivity threshold, the second light signal S2 may be emitted. The process can be repeated until the processor 110 has enough information with which to accurately characterize the fluidic content flowing through the conduit 250. In this respect, the implementations also provide for HDR sensing, as the travel paths between the various combinations of emitters 232 and detectors 234 are partially or entirely different.

The sensor data may then be provided to the processor 110, and the processor 110 may execute the fluid characterization algorithm or model, such as a neural network, Gaussian regression model, parametric model, or other machine learning model, trained with representative test data. In an exemplary implementation, the fluid characterization algorithm is a parametric model based on a data set machine-trained on one or more neural networks. For example, artificial intelligence may employ parametric models in order to determine what property of the fluidic content correlates to the signal values by the several detectors 234. More specifically, the fluidic content may be characterized as having a specific blood concentration by the processor 110 based on a known relationship between blood concentration and the effect on the light signals caused by certain blood concentrations. In another example, the processor 110 may have access to known relationships between signal attenuation and material properties to characterize the fluidic content. In yet another example, the processor 110 may have access to known relationships between signal timing and material properties to characterize the fluidic content. The relationships may be utilized alone or in combination. The known relationships may be developed by training the parametric model (or other machine learning models) with training data. Once the processor 110 has access to the known relationships, the processor 110 can characterize the fluidic content by providing signal emission and signal detection characteristics (along with the other characteristics described herein) as inputs to the parametric model.

In another implementation, the fluid characterization algorithm may include algorithmic modules disclosed in commonly-owned U.S. Patent Publication No. 2022/0008637, published Jan. 13, 2022, the entire contents of which are hereby incorporated by reference. The fluid characterization algorithm may include a feature extraction module to access the digital signal from the processor as the input and return a set of digital signals that represent one or more distinctive characteristics of the fluid for algorithmic analysis. A fluid motion model module may estimate the flow of the fluidic content when there are no strong features to track during a session of the fluid flow, such as during laminar or continuous patches of fluid flow. The fluid characterization algorithm may further include an optical mass estimation module to analyze the measured substance in the fluidic content when the fluidic content is passing through the conduit paired with the sensor module with detectors. A fluid scattering estimation module may determine the presence of scattering particles, which may be performed as part of estimating hemoglobin concentration in blood at different hemolysis levels that cause variations in scattering parameters. The fluid characterization algorithm may further include a fluid type classification module to classify the fluidic contents (e.g., determine a fluid type of the fluidic contents) within a given time frame. The fluid type classification module automatically categorizes different fluidic content with different properties based on the output of the sensor module or other measuring modality. A sensor merging module may combine measurement of measured substance between different sensors (e.g., with different measuring modalities or with different emitter-detector arrangements). Additionally or alternatively, the processor 110 may utilize known relationships between signal attenuation and material properties to characterize the fluidic content. For example, the processor 110 may use the Beer-Lambert Law to characterize the fluidic content based on the wavelength of the light signal and the attenuation of said signal, the attenuation known based on the strength of the light signal as received by one of the detectors 234. The processor 110 may also/alternatively perform other forms of spectroscopic analysis. For example, the processor 110 may perform spectroscopic analysis as described in the aforementioned United States Patent Publication No. 2022/0008637. Other mathematical phenomena are also contemplated.

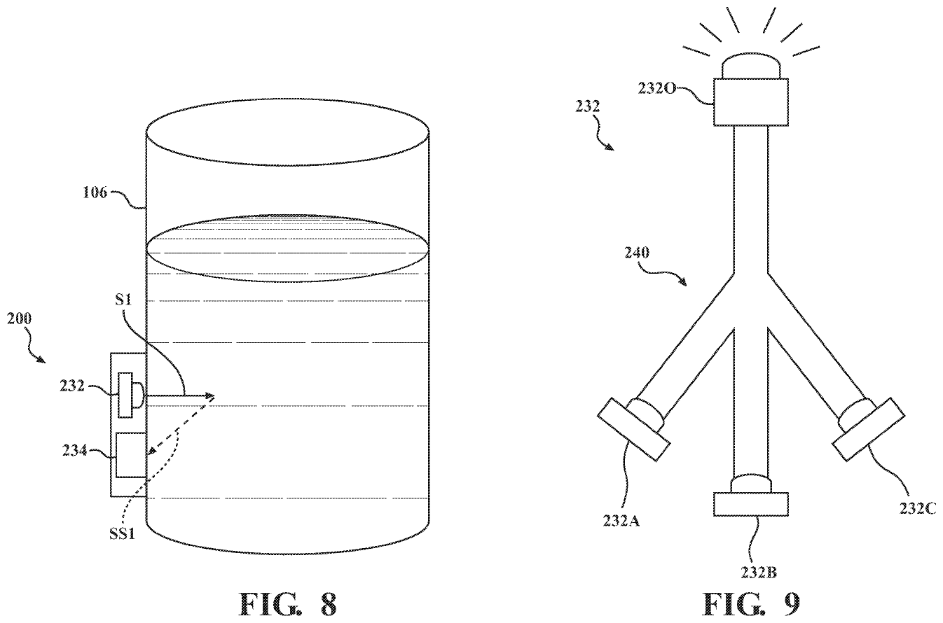

Referring now to FIG. 8, an alternative embodiment is provided in which the sensor module 200 is coupled to the receptacle 106, such as the receptacle 106 of the medical waste collection system 112. The sensor module 200 is illustrated with a single emitter 232 and a single detector 234 arranged adjacent to the emitter 232, but more than one emitter or detector may be provided. The sensor module 200 coupled to an outer wall of the receptacle 106 an external to a volume defined by the same. The arrangement of the emitter 232 and the detector 234, for example, being positioned adjacent to one another and to the outer wall, is configured to enable reflectance spectroscopy. In such an implementation, the light signals are directed at the fluidic content within the receptacle 106, wherein some of the light is absorbed and some is reflected. The first light signal S1 is shown being emitted into the receptacle 106, and the first scatter signal SS1 is shown reflecting back towards the detector 234. Analysis of the amount of absorbed light and scattered light, as received by the detector 234, may be used by the processor 110 to characterize the fluidic content. More specifically, the first scatter signal SS1 may contain multiple wavelength of light, and the intensity of each wavelength as received by the detector 234 corresponds to the absorbance and reflectance spectra of the fluidic content. If the fluidic content contains known materials, such as blood, the absorbance and reflectance spectra of the known material(s) may be considered by the processor 110 in order to determine the concentration of the known material(s) according to the algorithms disclosed herein.

The reflectance spectroscopy embodiment of FIG. 8 may be useful where the fluid in the receptacle 106 has high absorbance and scattering properties (e.g., a high concentration of blood), or where the receptacle 106 is too wide for the light signal from the sensor module 200 to traverse. Since the light signals emitted into the receptacle 106 could be entirely absorbed and/or scattered by the fluidic content before it could traverse the entire container, the detector 234 may be placed close to the emitter 232 so as to receive the scattered portion of the light signal (e.g., the first scatter signal SS1 from the first signal S1). The scatter signal SS1 as received by the detector 234 can be used by the processor 110 to characterize the fluidic content according to any of the methods described herein.

FIG. 9 illustrates an implementation of the emitter 232. In situations where the fluidic content is highly heterogeneous, the fluid may contain pockets of high and low concentrations of patient fluid, such as blood. The emitter 232 includes a light guide 240 disposed between the originating emitter 2320, and a plurality of LEDs 232A, 232B, 232C. The LEDs 232A, 232B, 232C may have different wavelengths. For example, a first LED 232A may be blue, a second LED 232B may be green, and a third LED 232C may be red. More than three LEDs may be provided, and alternative colors are contemplated. The LEDs 232A, 232B, 232C may be activated simultaneously or sequentially. A particular benefit is having the different wavelengths be output from the same location, i.e., the originating emitter 2320. As such, the light signals, despite being different wavelengths, have the same travel path through the fluids. The absorbance and/or scatter properties of the fluidic content may be more accurately determined by the processor 110 by, for example, eliminating instances in which different origins result in the light signals travelling through “pockets” of high and low concentrations of blood within the fluids in highly heterogeneous fluid. It is understood that the emitter 232 of FIG. 9 may be implemented as any one or more of the emitters 232 of the other implementations disclosed herein.

Several implementations have been discussed in the foregoing description. However, the implementations discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described. Although much of the above description references blood concentration, it will be appreciated that the sensor module 200 and its associated methods may be used to characterize fluidic content based on any passage of any fluid through the conduit 250. For example, when the fluid is a more complicated mixture, for instance blood where there is absorbance and light scattering due to the presence of cells, the multi-dimensional data within the rich data matrix (D′) may facilitate the determination of additional compounding variables such as the level of hemolysis in the blood (i.e., where cells rupture and their contents leak into the solution). This varying characteristic of blood can make determining its concentration difficult in existing systems with a single emitter-detector pair.

Exemplary systems for implementing the methods described herein may include a computing device (e.g., a smart phone, a tablet computer, or a wearable device) including the processor 110, and memory. As used herein, the term “memory” refers to a machine-readable medium able to store data temporarily or permanently and may be taken to include, but not be limited to, random-access memory (RAM), read-only memory (ROM), buffer memory, flash memory, and cache memory. The term “machine-readable medium” shall also be taken to include any medium, or combination of multiple media, that is capable of carrying (e.g., storing or communicating) the instructions for execution by the machine, such that the instructions, when executed by one or more processors of the system 100 (e.g., processor), cause the machine to perform any one or more of the methodologies described herein, in whole or in part. The term “machine-readable medium” shall accordingly be taken to include, but not be limited to, one or more tangible and non-transitory data repositories (e.g., data volumes). A “non-transitory” machine-readable medium, as used herein, specifically excludes propagating signals per se.

The various operations of example methods described herein may be performed, at least partially, by one or more processors that are temporarily configured (e.g., by software) or permanently configured to perform the relevant operations. Moreover, such one or more processors may perform operations in a “cloud computing” environment or as a service (e.g., within a “software as a service” (SaaS) implementation). At least some operations within any one or more of the methods discussed herein may be performed by a group of computers (e.g., as examples of machines that include processors), with these operations being accessible via a network (e.g., the Internet) and via one or more appropriate interfaces (e.g., an application program interface (API)). These and other variations, modifications, additions, and improvements fall within the scope of the subject matter herein.

Further inventive aspects are disclosed in the following exemplary clauses:

Clause 1—A medical waste collection system comprising: a vacuum pump; a receptacle in fluid communication with the vacuum pump and comprising an outer wall, wherein the receptacle is configured to collect fluids under influence of suction from the vacuum pump; a sensor module comprising a housing, an emitter and a detector, wherein the emitter and the detector are positioned adjacent to the outer wall and external to the receptacle, wherein the emitter is configured to emit light signals and the detector is configured to detect the light signals being absorbed and/or scattered by the fluid within the receptacle; and a processor in electronic communication with the sensor module and configured to receive sensor data from the detector and characterize a fluidic component of the fluid.

Clause 2—A sensor module for characterizing fluids from a patient, the sensor module comprising: a housing configured to be coupled to a non-circular conduit, the housing including: a first conduit seat arranged to be positioned adjacent to one side of the non-circular conduit when the housing is coupled to the non-circular conduit, a second conduit seat arranged to be positioned adjacent to an opposing side of the non-circular conduit when the housing is coupled to the non-circular conduit, and a lumen defined by the first and second conduit seats when the housing is coupled to the non-circular conduit, the lumen including: a minor cross-sectional dimension, and a major cross-sectional dimension which is larger than the minor cross-sectional dimension; a first light emitting diode coupled to the housing and configured to output light signals of a first wavelength; a second light emitting diode coupled to the housing and configured to output light signals of a second wavelength that is different from the first wavelength; a first detector coupled to the housing opposite the first light emitting diode about the minor cross-sectional dimension, the first detector being configured to detect the light signals of the first wavelength from the first light emitting diode and scattered light signals of the second wavelength from the second light emitting diode; and a second detector coupled to the housing opposite the second light emitting diode about the major cross-sectional dimension and configured to detect the light signals of the second wavelength from the second light emitting diode and scattered light signals of the first wavelength from the first light emitting diode.

Clause 3—The sensor module of claim 2, further comprising a processor in communication with at least one of the first light emitting diode, the second light emitting diode, the first detector, and the second detector.

Clause 4—The sensor module of claim 3, wherein the processor is configured to determine a concentration of a fluidic component of fluid flowing through the non-circular conduit based on the light signals as detected by the first and second detectors and the scattered light signals as detected by the first and second detectors.

Clause 5—The sensor module of claim 4, wherein the processor is further configured to determine the concentration of the fluidic component of the fluid flowing through the non-circular conduit by analyzing the light signals and the scattered light signals with a parametric model generated by a machine-trained neural network.

Clause 6—The sensor module of claim 3, wherein the processor is further configured to: determine an absorbance value and a scatter value from data from each of the first and second optical detectors for each of the first light signal, the second light signal, and the scattered light signals to provide a data matrix; and determine a concentration of a fluidic component within fluid flowing through the non-circular conduit based on the data matrix of the absorbance values and scatter values.

Clause 7—An optical emitter for use with a sensor module for characterizing fluids from a patient, the optical emitter comprising: an originating emitter configured to output light signals of multiple wavelengths; a first light emitting diode configured to output light signals of a first of the multiple wavelengths; a second light emitting diode configured to output light signals of a second of the multiple wavelengths; and a light guide coupling each of the first and second light emitting diodes to the originating emitter.

1. A method of characterizing fluids flowing through a non-circular conduit with a system including first and second optical emitters, first and second optical detectors, and a processor, the method comprising:
transmitting, with the first optical emitter, a first light signal through the non-circular conduit and the fluids along a first axis;
detecting, with the first optical detector, the first light signal that was at least partially absorbed by the fluids;
transmitting, with the second optical emitter, a second light signal through the non-circular conduit along a second axis different than the first axis such that a relative path of one of the first and second light signals through the non-circular conduit is shorter than the other;
detecting, with the second optical detector, the second light signal that was at least partially absorbed by the fluids; and
determining, with the processor, a concentration of a fluidic component within the fluids based on the first and second light signals.

2. The method of claim 1, wherein the first axis and the second axis are perpendicular to one another.

3. The method of claim 1, wherein the first axis and the second axis are transverse to a longitudinal axis of the non-circular conduit.

4. The method of claim 3, wherein the first and second axes correspond to a respective one of a major cross-sectional dimension and a minor cross-sectional dimension of the non-circular conduit.

5. (canceled)

6. The method of claim 1, wherein the step of transmitting the second light signal is performed after the step of transmitting the first light signal such that only a singular one of the first optical emitter and the second optical emitter is operating at a time.

7. The method of claim 1, further comprising alternating transmission between the first light signal and the second light signal.

8. The method of claim 7, wherein the step of alternating transmission between the first light signal and the second light signal is performed continuously and repeatedly during operation of the system.

9. The method of claim 6, further comprising:
comparing with the processor the first light signal against a sensor sensitivity threshold; and
performing the step of transmitting the second light signal in response to the first light signal being below the sensor sensitivity threshold.

10. The method of claim 1, wherein the step of determining the concentration of the fluidic component further comprises analyzing the first and second light signals with a parametric model generated by a machine-trained neural network.

11. The method of claim 1, further comprising:
generating, with the processor, a first scattered light value indicative of the first light signal as at least partially scattered by the fluids as detected by the second optical detector;
generating, with the processor, a second scattered light value indicative of the second light signal as at least partially scattered by the fluids as detected by the first optical detector; and
determining with the processor the concentration of the fluidic component further based on the first and second scattered light values.

12. (canceled)

13. A method of characterizing fluids flowing through a conduit with a system including first and second optical emitters, first and second optical detectors, and a processor, the method comprising:
transmitting, with the first optical emitter, a first light signal through the conduit and the fluids;
transmitting, with the second optical emitter, a second light signal through the conduit and the fluids;
detecting, with each of the first and second optical detectors, the first and second light signals that were at least partially absorbed and scattered by the fluids, wherein the first and second optical emitters and the first and second optical detectors are arranged in an array about the conduit such that distances between combinations of the first and second optical emitters and the first and second optical detectors are different;
determining with the processor an absorbance value and a scatter value from data from each of the first and second optical detectors for each of the first and second light signals to provide a data matrix; and
determining with the processor a concentration of a fluidic component within the fluids based on the data matrix of the absorbance values and scatter values.

14. The method of claim 13, further comprising:
transmitting the first light signal at a first wavelength; and
transmitting the second light signal at a second wavelength different than the first wavelength.

15. The method of claim 13, wherein the step of transmitting the second light signal is performed after the step of transmitting the first light signal such that only a singular one of the first optical emitter and the second optical emitter is operating at a time.

16. The method of claim 13, further comprising alternating transmission between the first light signal and the second light signal.

17. The method of claim 16, wherein the step of alternating transmission between the first light signal and the second light signal is performed continuously and repeatedly during operation of the system.

18. A method of characterizing fluids flowing through a conduit with a system including optical emitters arranged in an array about the conduit, optical detectors arranged in the array about the conduit, and a processor, the method comprising:
transmitting, with the optical emitters, light signals through the conduit and the fluids, wherein the optical emitters are sequentially activated, one at a time, in a positional order about the conduit;
detecting, with the optical detectors, the light signals that were at least partially absorbed and scattered by the fluids;
determining with the processor an absorbance value and a scatter value from data from the optical detectors for each of the light signals; and
determining with the processor a concentration of a fluidic component within the fluids based on the absorbance value and scatter value.

19. The method of claim 18, wherein the optical emitters and the optical detectors are arranged in an array about the conduit such that distances between combinations of the optical emitters and the optical detectors are different.

20. (canceled)

21. The method of claim 14, wherein the step of determining the concentration of the fluidic component further comprises analyzing the first and second light signals with a parametric model generated by a machine-trained neural network.

22. The method of claim 14, wherein the conduit is non-circular.

23. (canceled)

24. The method of claim 23, wherein the optical detectors are arranged at different angles relative to each of the optical emitters.

25. The method of claim 1, further comprising displaying on a display the concentration of a fluidic component.

26. (canceled)

27. (canceled)
